(19) United States
(12) Patent Application Publication
(10) Pub. No.: US 2018/0358122 A1
(43) Pub. Date: Dec. 13, 2018

(54) SYSTEM, DEVICE AND METHOD FOR GUIDING A PATIENT IN A HOSPITAL SETUP

(71) Applicant: KONINKLIJKE PHILIPS N.V., EINDHOVEN (NL)

(72) Inventors: Prasad RAGHOTHAM VENKAT, Bangalore (IN); Meru Adagouda PATIL, Bangalore (IN); Nagaraju BUSSA, Bangalore (IN)

(73) Assignee: Koninklijke Philips N.V., Eindhoven (NL)

(21) Appl. No.: 15/778,857

(22) PCT Filed: Dec. 21, 2016
(86) PCT No.: PCT/EP2016/082168

§ 371 Date: May 24, 2018

(30) Foreign Application Priority Data
Dec. 21, 2015 (IN) 6799/CHE/2015

(51) Int. Cl. G16H 40/20; G16H 15/00; G16H 10/60; G01C 21/20

(52) U.S. Cl. CPC G16H 40/20; G01C 21/206; G16H 10/60; G16H 15/00

(57) ABSTRACT

Systems, methods and devices for guiding a patient through events in a hospital and recording information related to the events. The patient is guided through the hospital by scheduling events for a patient based on a scheduling model and at least one scheduling parameter, providing navigation information to the patient based on the scheduling of the events to enable performing the event and generating a report of the events.

### FIELD

This disclosure relates to guiding a patient in a hospital setup, particularly a system, device and method for scheduling of events, assisting in navigating through the hospital setup, and recording the events and conversations occurring between a patient and resources for effective reporting later on.

### BACKGROUND

Generally, a patient is engaged with various events in a hospital. These events can include, but are not limited to consulting a doctor, undergoing a lab test, diagnosis, registration at the hospital, making payments, generating reports etc. These events may follow a particular sequence or may be changed based on the criticality and dependencies with other events. Also, one or more of these events may recur. A patient is currently required to constantly engage with the hospital resources such as staff, labs, pharmacy, etc., and has to be guided from time to time. The patient may be required to physically move from one location to another within a hospital to achieve the purpose of making a visit to the hospital. This involves time and engagement by the patient as well as the hospital resources. This uncertainty can lead to confusion by the patient and long wait times. Also, there may exist uncertainty in guidance provided to the patient, due to a lack of coordination between the patient's planned events as one event may have a significant impact on other events.

Also, if there is a change in the event, the engagement sequence may undergo a change, based on which the resource availability needs to be considered accordingly. This may have a significant impact on the time that is being spent, and may involve manual intervention to handle the situation. For example, a patient may be scheduled to receive a treatment for a condition, but findings in an x-ray show that the issue is different from that what was originally thought such that the patient needs a different or additional treatment. For example, the patient may now need an MRI, which has not been previously scheduled. Currently, the patient would have to interact with the hospital staff (and possibly multiple members of the hospital staff) to schedule the MRI. The patient may also have to cancel the other unnecessary treatment. If the patient does not do this, the hospital resource may remain unused during the time that the patient was scheduled to use that resource. Thus, there is time wasted for the patient, the hospital staff and the hospital resources.

There may be a great deal of interaction taking place between the hospital resources and the patient at various steps in the schedule. Unless these interactions are captured, important information regarding patient's health, physician's observations and other history information may be lost.

Currently, the guidance to the patient is provided on an ad-hoc basis, at the most considering the hospital resources. For example, the hospital may have a radiology information system (“RIS”) that is used to manage the imaging department, including, such things as patient scheduling and resource management. Thus, in the example above, where the patient needs an MRI, the patient may see a member of the hospital staff that may access the RIS and provide the patient with an appointment for the MRI. However, this is not optimal, because the patient has to deal with a member of the hospital staff to make the appointment and it does not address the patient at a personalized level, e.g., considering the patient profile and possibly other factors. For example, how does this rescheduling effect the other scheduled events for the patient, does the patient need to come back on a different day, does the MRI overlap with a non-imaging event, were other events properly cancelled, etc. In addition, it is not optimal for the hospital staff because they have to manually account for these other factors. Also, health data relating to similar or relative health conditions or patient profiles are not being considered for this purpose.

Therefore, there exists a need for a solution that can schedule the events optimally and if required reschedule the events, and also provide recommendations on performing additional tests or alter the events, and also capture interactions at every level for providing better clinical care.

### SUMMARY

The exemplary embodiments relate to systems, methods and devices for scheduling of events, assisting a patient in navigating through the hospital for the events and recording the results of the events.

According to one aspect of the present disclosure, a method is provided for guiding a patient in a hospital. The method includes scheduling events for a patient based on a scheduling model and at least one scheduling parameter, providing navigation information to the patient based on the scheduling of the events to enable performing the events and generating a report of the events.

According to another aspect of the present disclosure, a system is provided for guiding a patient in a hospital. The system includes a scheduling unit configured to schedule events for a patient based on a scheduling model and at least one scheduling parameter, a navigation unit configured to provide navigation information to the patient based on the scheduling of the events to enable performing the events and a report generating unit configured to generate a report of the events.

According to further aspect of the present disclosure, a system is provided for guiding a patient in a hospital. The system includes a server device configured to extract information from one of a patient record and a hospital resource to generate a list of events for a patient, the server device further configured to generate a schedule for the patient based on the list of events. The system further includes a user device configured to receive the schedule from the server device and navigate the patient to a first event on the schedule.

### BRIEF DESCRIPTION

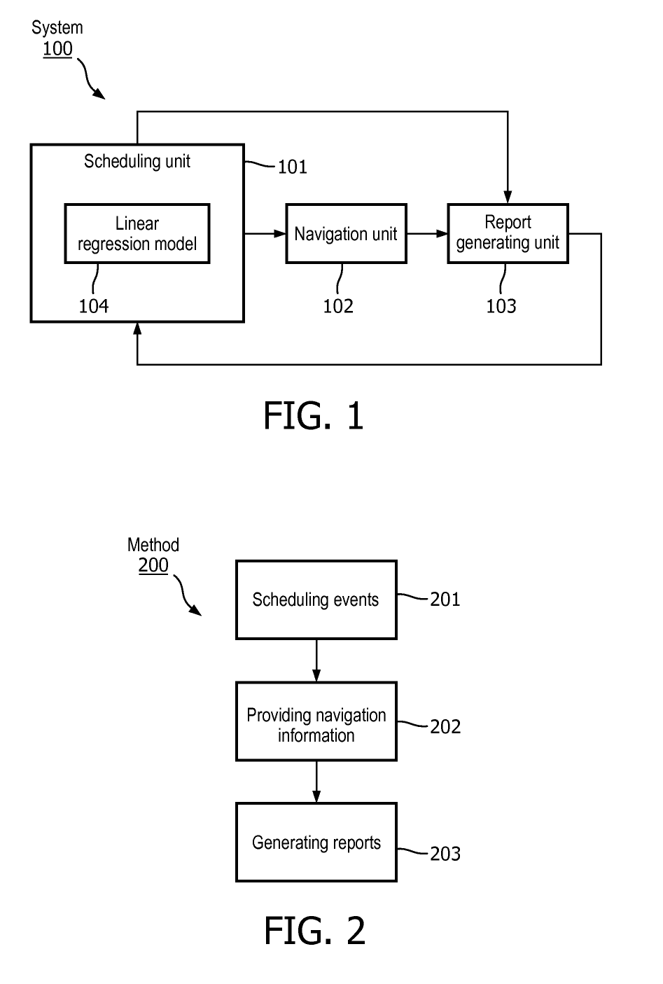

FIG. 1 shows an exemplary system for guiding a patient in a hospital according to various exemplary embodiments described herein.

FIG. 2 shows an exemplary method for guiding a patient in a hospital according to various exemplary embodiments described herein.

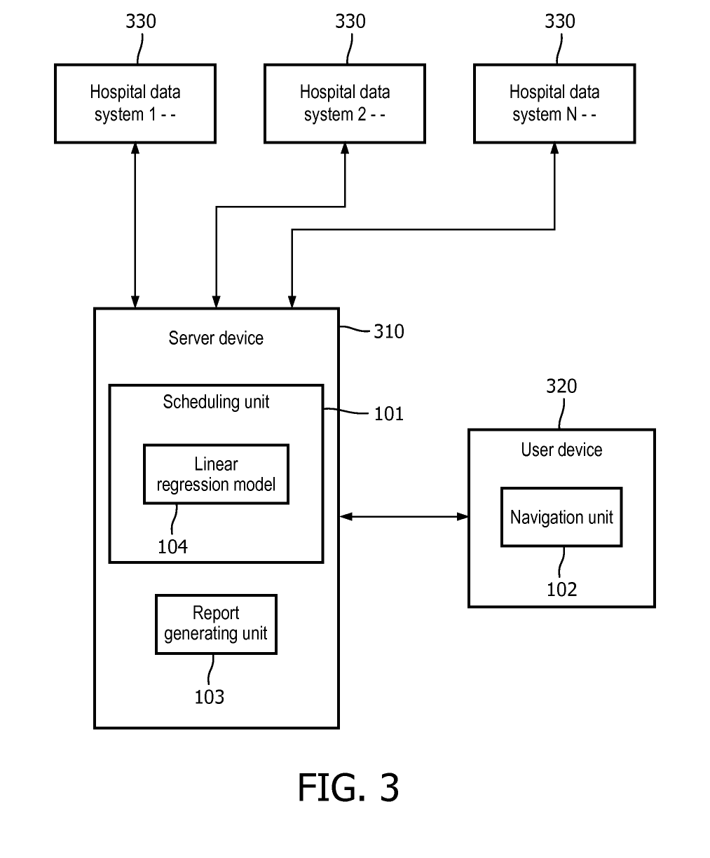

FIG. 3 shows a further exemplary system for guiding a patient in a hospital according to various exemplary embodiments described herein.

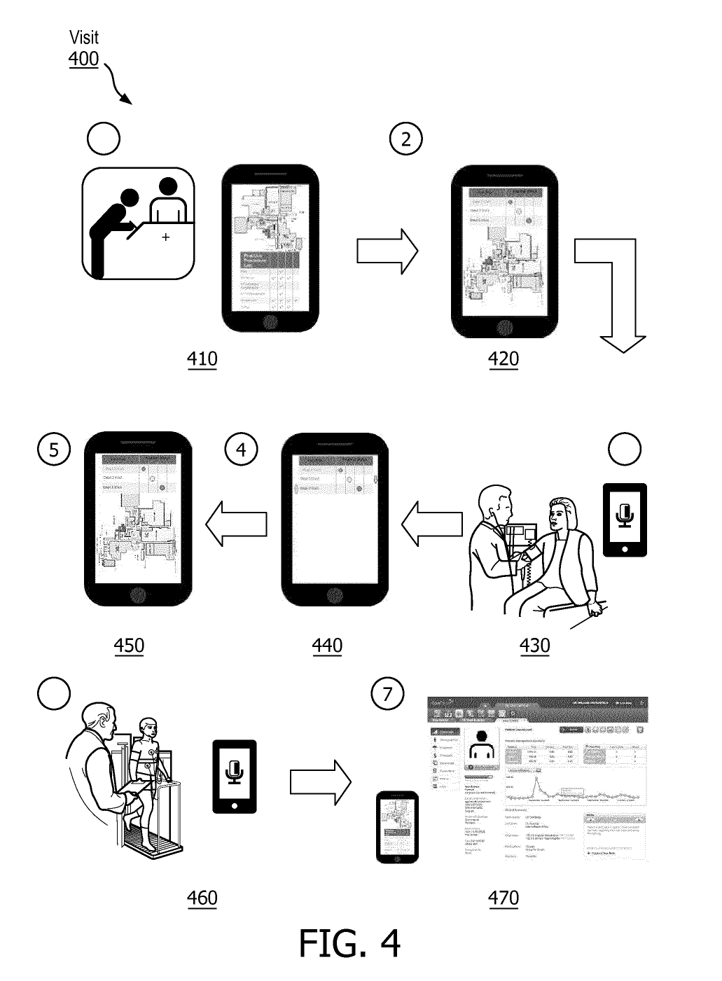

FIG. 4 shows the exemplary system guiding a patient through a typical hospital visit according to various exemplary embodiments described herein.

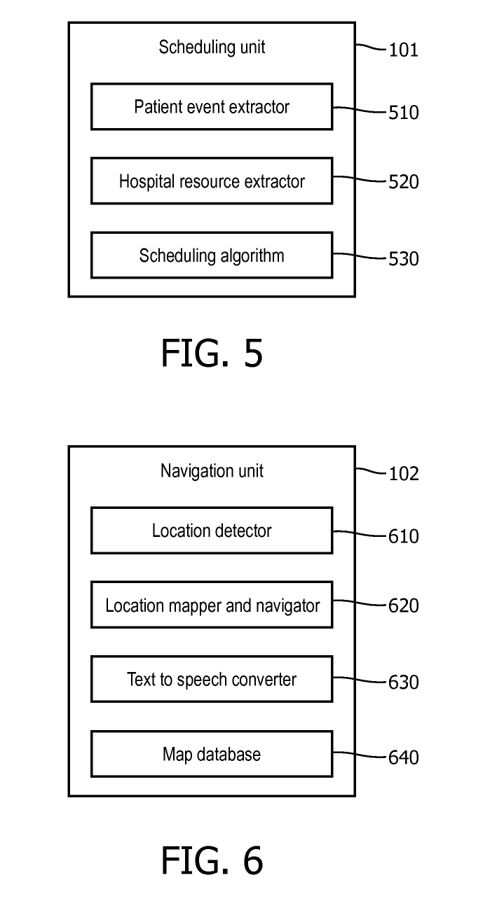

FIG. 5 shows an exemplary embodiment of the scheduling unit according to various exemplary embodiments described herein.

FIG. 6 shows an exemplary embodiment of the navigation unit according to various exemplary embodiments described herein.

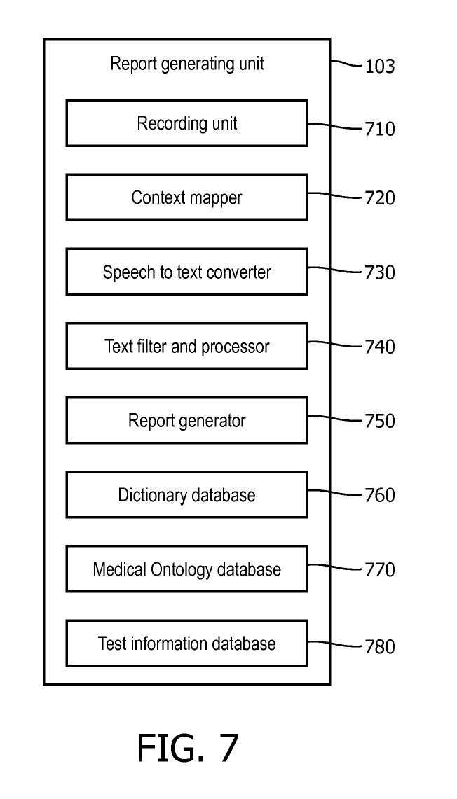

FIG. 7 shows an exemplary embodiment of the report generating unit according to various exemplary embodiments described herein.

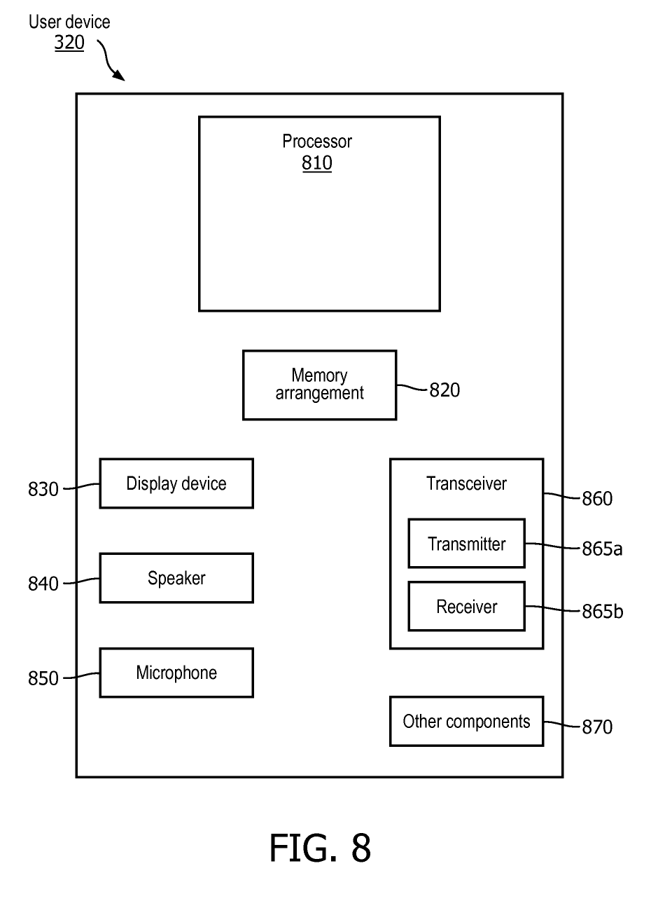

FIG. 8 shows an exemplary user device according to various exemplary embodiments described herein.

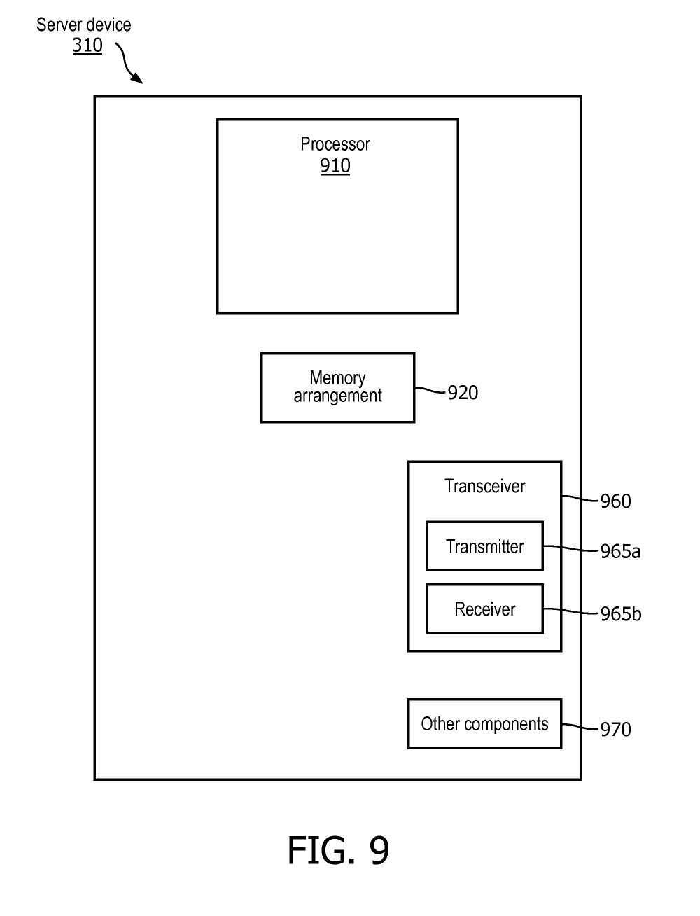

FIG. 9 shows an exemplary server device according to various exemplary embodiments described herein.

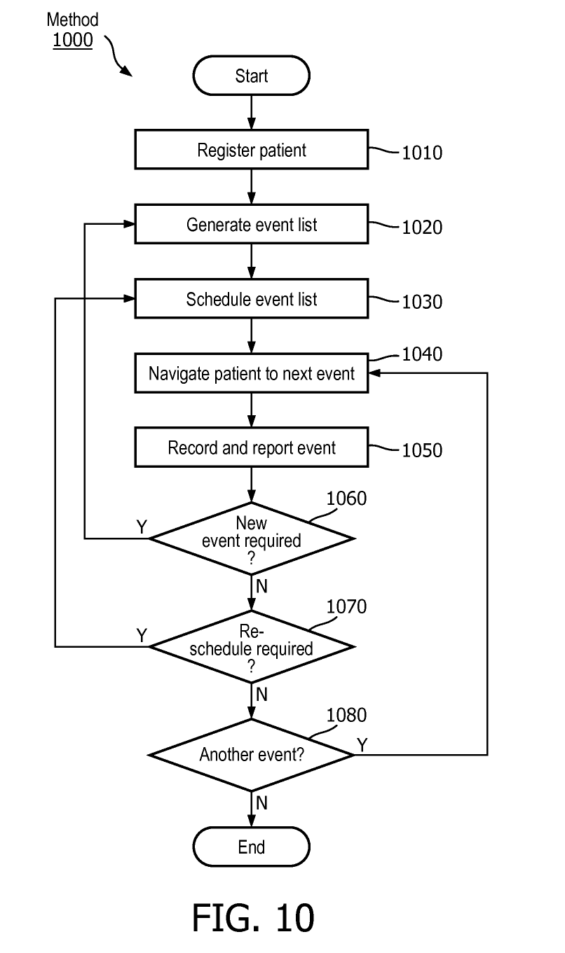

FIG. 10 shows another exemplary method for guiding a patient in a hospital according to various exemplary embodiments described herein.

### DETAILED DESCRIPTION

The exemplary embodiments may be further understood with reference to the following description and the appended drawings wherein like elements are referred to with the same reference numerals. The exemplary embodiments relate to a system, device and method for assisting a patient in navigating through a hospital setting. It is noted that this navigation through the hospital setting may include physical navigation such as providing the patient with directions to various locations within the hospital setting (e.g., x-ray lab, MRI lab, cardiac unit, exam rooms, pharmacy, etc.), scheduling navigation such as providing the patient with optimal times, locations and personnel for various diagnostic tests, patient personalization navigation such as providing the patient with their electronic medical record (EMR) that may accompany the patient and be updated as the patient navigates the hospital setting, etc. Other examples of patient navigation will be provided below.

In a typical hospital visit, a patient tends to follow various broad sequences of events. Examples of these broad sequences may include:

Consulting with doctor→Undergo screening/diagnostic tests→Consulting with doctor (same or different department)

Undergo screening/diagnostic tests→Consulting with doctor (same or different department)→Undergo screening/diagnostic tests

Consulting with doctor→Consulting with doctors from different department

Consulting with doctor→Undergo screening/diagnostic tests→In Patient Admission

Those skilled in the art will understand that the above sequences are only exemplary and there are many additional sequences that a patient may have to navigate when visiting the hospital. However, from this short list of examples, it can be seen that the patient may have many interactions with hospital staff and resources that the patient needs to navigate to have a successful and efficient visit.

FIGS. 1 and 2 show an overview of an exemplary system 100 and method 200, respectively, for guiding a patient in a hospital setup. The system 100 comprises a scheduling unit 101 for scheduling the events 201 for a patient. The scheduling unit 101 may be, for example, part of a Radiology Information System (“RIS”), medical imaging workstation, a Hospital Information System (“HIS”) or other suitable system in a hospital. The scheduling unit 101 schedules the events based on a linear regression model 104. The events are scheduled optimally based on at least one of scheduling parameters. The scheduling parameters may include resource availability, time for performing the event, distance, movement, waiting time, criticality of the event, patient profile, clinical data, clinical condition, other planned events, etc. The resource may include doctor, technician, specialist or any other resource that is related to the event.

The events may include activities such as lab tests, diagnostic tests, consultation with physicians, etc. These events may be of same type or different type and may or may not be directly related to each other. Also the events may take place or occur at one or more locations in a hospital.

The system further comprises a navigation unit 102 for providing navigation information 202 to the patient. The navigation information includes, but is not limited to, information or details regarding location of the facilities such as labs, doctor's station, navigation path between such facilities, time to navigate, resource availability and time of availability, sequence of events, sequence of navigation, type of activity that will be performed, basic knowledge about event, etc. The navigation unit 102 may have audio and video capability so as to provide guidance to the patient through an audio or video interface. Also, the navigation unit 102 may have location detection capability enabled through Global Positioning System (GPS), WLAN triangulation, or the like to provide the location of the patient, in real time. The navigation information is provided based on the schedule of events provided by the scheduling unit 101.

Further, the system 100 has a report generating unit 103 for generating reports 203. Generating reports includes recording the interaction between the patient and the resource such as doctor, technician, specialist etc. It also includes providing recommendations on events such as altering the events, altering the schedule of events or the like, to the patient. These recommendations may be provided based on one or more of patient profile, clinical condition of the patient, other patient profile and/or clinical condition related to the patient profile and clinical condition etc.

Each of the components of the system 100 and the steps of the method 200 will be described in greater detail below. The scheduling unit 101, navigation unit 102 and the report generating unit 103 are processing units which have the capability to process data or information. Also, they may be integrated together in possible combination. The methods described herein may be performed automatically, and offline or in real time. These three units help in providing guidance to a patient across hospital departments, optimize utilization of hospital resources and automatically generate a patient record/discharge summary.

FIG. 3 shows a further exemplary system 300 for guiding a patient in a hospital according to various exemplary embodiments described herein. The system 300 comprises a server device 310 and a user device 320. Exemplary implementations of the server device 310 and the user device 320 will be described in greater detail below. However, in general, the user device 320 may be described as a portable device that can be handed to the patient or a person accompanying the patient to a hospital visit. The user device 320 is connected to the hospital resources and can make all the relevant information—patient information, hospital map or 3D Visualization, and next steps, where to go, what preparation is needed, etc. available to the patient or the caregiver with which the patient is interacting. The server device 310 may be described as a backend processing device that receives information from various hospital data systems 330 and processes information from these systems and the information received from the user device 320. The server device further distributes the processed information to the various systems 330 and user device 320 as needed.

In this exemplary embodiment, it may be considered that the server device 310 will implement the functionality of the scheduling unit 101 with the linear regression model 104 and the report generating unit 103. The user device 320 will implement the functionality of the navigation unit 102. However, it is noted that this distribution of the functionality between the devices 310 and 320 is only exemplary. It is possible that any one of the devices 310 and 320 may implement all the described functionalities or that the functionalities are split in a different manner or that each of the devices shares portions of the described functionality, e.g., the server device 310 implements a first portion of the navigation unit 102 functionality and the user device 320 implements a second portion of the navigation unit 102 functionality.

As shown in FIG. 3, the server device 310 and the user device 320 include a two-way communication path such that each device may communicate information to the other device. In one example, each of the devices 310 and 320 may be connected to a Local Area Network (LAN) that is hosted by the hospital. The devices 310 and 320 may communicate via the LAN. In a further example, the user device 320 may be a wireless device and the hospital LAN may include a Wireless LAN (WLAN) (e.g., a plurality of wireless access points (APs) distributed throughout the hospital facility) that allows the server device 310 to communicate wirelessly with the user device 320. Those skilled in the art will understand that there are also other manners and networks that may be used to communicate between the server device and user device, e.g., cellular networks, short-range networks (Bluetooth), etc.

FIG. 3 also shows that the server device 310 may receive input from other hospital data systems 330. These inputs from other hospital systems 330 may include, for example, a patient's electronic medical record (EMR), a map of the hospital, data from a diagnostic testing facility, etc. To provide a more specific example, it may be considered that one of the data systems 330 that is implemented by the hospital is an imaging system that stores information regarding imaging tests, such as x-rays, MRIs, etc. The imaging data system 330 may collect information for each of the imaging tests that are performed by the hospital. The information may include, for example, the type of test, the duration of the test, the expertise level of the technician, the expertise level/experience level of the doctor, patient information (e.g., Male/Female), etc.

As will be described in greater detail below, the server device 310 and the user device 320 may use the information that is input from the hospital data systems 330 in implementing the functionality associated with the scheduling unit 101, the navigation unit 102 and the report generating unit 103. It should also be noted that the hospital data systems 330 may also provide inputs directly to the user device 320, rather than the server device 310. The communication between the server device 310 and the hospital data systems 330 may also be two-way communications. For example, if the scheduling unit 101 of the exemplary embodiments modifies the schedule of a patient, the server device 310 may send this modified schedule information to the relevant hospital data system 330. The data that is input to the server device 310 and/or the user device 320 from the hospital data systems 330 may be stored locally, e.g., in a memory of the server device 310 and/or user device 320, or it may be retrieved from the hospital data systems 330 as needed.

FIG. 4 shows the exemplary system guiding a patient through an exemplary hospital visit 400 according to various exemplary embodiments described herein. In this example, it will be considered that the exemplary system 300 is the system in use that guides the patient through the hospital visit 400. However, as described above, the functionalities described for system 300 may be implemented in other manners.

In step 410, the patient may register at the hospital reception. Part of this registration may be providing identifying information to the receptionist such as a patient identifier. The patient identifier may be any type of an identification code, such as a Medical Record Number (MRN) or a Patient Identifier. The patient identifiers may also be stored in a database that allows for patient specific queries. It should also be understood that the database may represent a series of databases or other types of storage mechanisms that are distributed throughout system 300. During the registration step 410, the hospital staff configures a list of events for the patient. This list may be generated in a variety of manners. For example, based on the patient's past records available in the system (e.g., the patient may have come for follow up of previous visit), based on a referral from another caregiver (e.g., the patient may have specific information about the department(s) to be visited or tests to be performed), based on symptoms being presented by the patient (e.g., looking at the symptoms, the hospital staff may generate a primary event list), etc.

The patient will then be issued the user device 320 that will include the patient's initial schedule for the visit 400. As described above, the patient's visit will include a series of events that are scheduled for the patient and that will occur for the patient during the visit. Before the patient arrives, the system 300, via the scheduling unit 101, may set up the initial patient schedule for the visit 400.

For example, it may initially be considered that the patient has the following events to be scheduled for the visit 400, a consultation with a primary caregiver, an x-ray on the patient's arm and a follow-up consultation with an orthopedic physician. It should be understood that these events are only exemplary and are being used to illustrate the functionality of the system 300. An actual patient may have more or less events that are scheduled for a particular visit. These initial events may have been pre-arranged prior to the visit, thus the scheduling unit 101 may have generated the schedule prior to the patient's arrival. In another example, the scheduling unit 101 may generate the initial schedule when the patient arrives so as not to tie up hospital resources until it is verified the patient has arrived.

FIG. 5 shows an exemplary embodiment of the scheduling unit 101 according to various exemplary embodiments described herein. The scheduling unit 101 includes a patient event extractor 510, a hospital resource extractor 520 and a scheduling algorithm 530. As described above, in the system 300, the scheduling unit 101 may be implemented by the server device 310.

The patient event extractor 510 may extract a list of events corresponding to different department visits that the patient is supposed to undergo, from, for example, the hospital information system or the Electronic Medical Record (EMR) system. These exemplary systems may be one or more of the hospital data systems 330. Returning to the example, started above, the patient associated with the visit 400 has the following events that need to be scheduled, a consultation with a primary caregiver, an x-ray on the patient's arm and a follow-up consultation with an orthopedic physician. These events may be extracted by the patient event extractor 510 from the patient's EMR or other hospital data system 330 having information about the events for this particular patient. Based on this information, the scheduling unit 101 understands the events that need to be scheduled.

The hospital resource extractor 520 may extract a list of resources that are relevant to current patient and their current status. Continuing with the example started above, the hospital resource extractor 520 may extract the current schedules of the primary caregivers, the x-ray department and the orthopedic physicians. Again, the schedule for each of these hospital resources may be included in one or more of the hospital data systems 330. Based on the information extracted by the patient event extractor 510 and the hospital resource extractor 520, the scheduling unit 101, using the scheduling algorithm 530, may generate the patient's initial schedule.

It should be noted that the scheduling unit, in addition to the basic information described above, may also consider additional information related to the patient's events and hospital resources when generating the schedule. This additional information may include, for example, the load of the resources, clinically relevant sequences for tests, pre-requisite information about the tests, resource constraints like clinician leaving department for handling emergency cases, current technicians' expertise and experience based on the past data of scans/tests executed, clinicians expertise, experience and knowledge based on the past data of tests and consultation done, the lab/station device capability, additional resource availability, etc. This additional data may be used along with the data about the typical time taken for each consultation/test during the scheduling process.

For example, a model may be generated using the past data at a particular station/lab with the parameters such as type of test, duration of the test, expertise level of the technician, expertise level/experience level of the doctor, etc. The output may be the duration of the test. As described above, one exemplary scheduling algorithm 530 may use a linear regression model 104. The linear regression model 104 may be generated using the variables described above to identify the duration of the test. An exemplary linear regression model may be generated based on the following:

Y=b₀+Σ(bᵢXᵢ)+e
where Y is the test duration (dependent variable), and Xᵢ is a set of independent variables as defined above.

Based on the data from the above, the scheduling unit reserves the time on the respective test stations or labs. The scheduling unit 101 may also consider the other patients in the queue and shows the wait time at a particular station. It should be noted that a linear regression model is only example of a scheduling model and other models may be used.

Thus, at the completion of step 410, the patient has registered at the hospital and received the user device 320 that includes the initial schedule with the first scheduled event for the patient. The visit 400 may then process to step 420. In this step the patient may use the navigation unit 102 that is resident on the user device 320 to navigate to the first scheduled event.

FIG. 6 shows an exemplary embodiment of the navigation unit 102 according to various exemplary embodiments described herein. The navigation unit 102 includes a location detector 610, a location mapper and navigator 620, a text to speech converter 630 and a hospital map database 640. Again, in this exemplary embodiment, it is considered that the navigation unit 102 functionality is performed by the user device 320, but those skilled in the art will understand that some or all of the functionality may also be resident on the server device 310.

The location detector 610 determines, in real time, the current location of the patient based on the location of the user device 320. The location detector 610 may determine the current location based on, for example, the hospital wireless network (e.g., WiFi, etc.), a GPS chip in the user device 320, etc. Those skilled in the art will understand that there are various manners of determining the location of the user device 320 and any of these manners may be used.

The hospital map database 640 includes a hospital map or a 3D visualization of the hospital. The current location information may be provided to the location mapper and navigator 620, that uses the hospital map database 640 to position the current location of the patient in a map that is visually made available to the patient via a display device of the user device 320. The navigation unit 102 also communicates with the scheduling unit 101 to retrieve the patient event list to generate a list of navigation steps to direct the patient to the next scheduled location. The navigation steps may be presented to the user in any known manner, such as via text directions, via arrows shown on the map, via lines shown on the map, via audible commands, etc. Information about the navigation steps may also be provided to the text to speech converter 630, where it converts navigation steps into audio guidance to guide patient to next department where the next visit is planned. Thus, the user device 320 may include other input/output components such as a speaker for the user to hear the speech produced by the text to speech converter 630.

The navigation unit 102 may also correct the route if the patient has deviated from the path. For example, a display of the user device 320 may flash a red “X” or some other alert if the patient is going in the wrong direction. The navigation unit 102 may also display common areas that are relevant to the patient as a general guide, such as, locations of rest rooms, common areas, food points, other departments, emergency exits, etc. If the patient is deviating from the displayed route, the navigation unit 102 may cause the user device 320 to display a prompt to question whether the patient desires to deviate from the route, e.g., “is this a planned deviation?”, and then further query the patient about the reason for the deviation. In another example, a display of the user device 320 may display icons such as a rest room that the user may select so the navigation unit 102 may insert a rest room stop into the planned route.

Returning to FIG. 4, the navigation unit 102 has successfully navigated the patient to the first event which, in the example, is a consultation with a primary care giver. This is illustrated in step 430 of FIG. 4. When the patient arrives at the event, the report generating unit 103 may be initiated to record the actions that occur during the event in step 430, e.g., the consultation with a primary care giver. The initiation of the report generating unit 103 may be based on communication with the navigation unit 102. For example, when the patient arrives at the location for the event, this may initiate the report generating unit 103. In another example, the patient may provide the user device 320 to the primary caregiver who may manually initiate the report generating unit 103.

FIG. 7 shows an exemplary embodiment of the report generating unit 103 according to various exemplary embodiments described herein. The report generating unit 103 includes a recording unit 710, a context mapper 720, a speech to text converter 730, a text filter and processor 740, a report generator 750, a dictionary database 760, a medical ontology database 770 and a test information database 780. In this exemplary embodiment, it is considered that the report generating unit 103 functionality is performed jointly by the server device 310 and the user device 320 (even though FIG. 3 shows the report generating unit 103 resident on the server device 310). Those skilled in the art will understand that some or all of the functionality may be resident on any one of the devices 310 and 320.

One functionality of the report generating unit 103, when initiated, is to provide information to the patient. For example, when the patient arrives at the destination, the report generating unit 103 may cause the user device 320 to display information to the patient such as what is expected to be done by the patient, tips and other useful information about the test and possible outcomes of the test. This information may be included in the test information database 780, such that the report generating unit 103 extracts the information for the particular test and displays the information to the patient.

In addition, when the event commences (e.g., the consultation with the primary care giver), the recording unit 710 of the report generating unit 103 may be started and stopped as required. The recording unit 710 may use information such as location information from navigation unit 102, the patient event list from scheduling unit 101, time-stamp information and actions received from the caregiver, e.g., patient detail entry into the user device 320, etc., to deduce when to start and stop the recording unit. This will help in recording only the relevant information and avoids unnecessary recording of noise that may be captured when patient is moving from one department to other. It should be noted that the recording unit 710 may record the conversation using a recording device such as a microphone of the user device 320. The conversation may then be stored locally at the user device (e.g., in a memory of the user device 320) or the user device may transmit the conversation to be stored at the server device 310.

After recording the relevant conversation, the report generating unit 103 processes the conversation using Natural Language Processing (NLP) techniques, for example, Lexical Answer Type (LAT), Relation Detection and decomposition of the conversation. Besides this, the report generating unit 103 performs a keyword search and/or responsive coding on the conversation data to identify the relevant text and the intent of the conversation. Based on this, the direction of the conversation is detected and may be used for various purposes.

In one example, the conversation may be used to record the event in the patient's medical record, e.g., EMR. For example, the context mapper 720 may use a time-stamp of the recording chunks along with patient event list to map caregiver action/entries in hospital information system with the recording files. These files may then be passed to the speech to text converter 730 that may use a dictionary to convert the speech into text. The report generating unit 103 may include a dictionary database 760 to perform this functionality. The text information may then be passed to the text filter and processor 740 that may use medical ontology to filter unwanted text and make text content clinically relevant. The report generating unit 103 may include a medical ontology database 760 such as UMLS or SNOMED to perform this functionality. Finally, the report generator 750 may use the cleaned text along with caregiver entered information such as clinical findings, laboratory tests, etc. to generate a patient report/discharge summary. This information may then be recorded into the patient's EMR. Again, depending on the particular implementation, these various steps may be performed at the user device 320 or the server device 310. Thus, in this example, the system 300 has captured the actions that have occurred for the first event and has recorded the actions into the patient's EMR for the event that may now be distributed downstream for future events.

In the above example, it can be seen that when the patient, during and after the tests, consults the physician or technician the user device 320 may record all relevant conversations and patient-physician interaction. This recording will contain information related to the disease detection, symptoms, related queries and responses, next steps, when to come for follow up, life style changes suggested, etc. The above-described process of capturing the interaction and electronically storing the interaction in both audio and text files (e.g., EMR) ensures that loss of information is minimized. In addition, the recollection of the interaction is no longer dependent on what the patient perceives and interprets as the information.

In another example, the report generating unit 103 may use the recorded conversation to determine whether there is a need for any additional tests other than the ones currently scheduled are determined. If so, the report generating unit 103 may add this additional test to the list and may also seek the physician (e.g., primary caregiver) to acknowledge the new test. The report generating unit 103 may identify new tests using data analytics. For example, the report generating unit 103 may access a patient database to identify one or more similar patients to the current patient. Similarities may be based on, for example, the age, condition, list of tests prescribed, lab values of the tests, etc. The report generating unit 103 may then check if there are any other definitive tests that were prescribed to other similar patients and also whether those tests were beneficial for the patients. Based on this, the report generating unit 103 may provide a suggestion for the physician to add particular test(s) to the list.

To provide a specific example, the report generating unit 103 may identify that the patient is complaining of arm pain, which is why the X-ray on the arm was initially scheduled. However, the report generating unit 103, based on the conversation between the patient and the primary caregiver and the comparison of similar patients, may recommend a stress test for the patient because arm pain may also be an indicator of cardiac issues. The report generating unit 103 may add the stress test to the list of events for the patient and prompt the primary caregiver, via a display on the user device 320, as to whether the stress test should be added to the list of events. In another exemplary embodiment, rather than a recommendation by the report generating unit 103, the primary caregiver may make the recommendation of the stress test and the recording and processing of the conversation by the report generating unit 103 may cause the stress test to be included in the list of events.

This new event may be then communicated by the report generating unit 103 to the scheduling unit 101 because the new event needs to be scheduled. In addition, the scheduling unit 101 may need to reprioritize the pending tests based on the definitiveness of the outcome of the previous events. For example, the newly prescribed stress test may have a higher priority than the previously prescribed x-ray. In another example, the scheduling unit 101 may reshuffle the schedule because the new test (or previously scheduled tests) have a reduced wait time. In such a situation, the initial schedule may be updated to account for the new test or change in scheduling priorities. If at any given situation there is a possibility of getting either of the tests done based on the wait time, the scheduling unit 101 may decide on the more definitive test to be done earlier than the other.

In another example, the hospital may have multiple labs or stations of the same kind. For example, if it were considered that the patient still had to have the x-ray performed, but the current x-ray station was backed up for some reason, the system 300 may determine that a second different x-ray station should be used for the patient. From this example, it should be seen that the system 300 may be constantly updated as to the current schedule and throughput of the particular stations so that such a determination may be made.

The system 300 may use of historical data of the stations along with other parameters such as the device capability, the technician's past experience for this particular test, the clinician's past expertise for this particular test, the patient's condition (age, gender, etc.), patient's physical condition, pre-existing diseases, in-patient/out-patient, any other external parameters that are relevant. This information may be used by the scheduling unit to generate an output that determines if the station is suitable or not. In one example, a logistic regression model may be used to check the suitability of the stations:

$$P ( Y = 1 ) = \frac{1}{1 + {e}^{- ( {b}_{0} + \sum ( {b}_{i} {X}_{i} ) )}}$$

where Xᵢ is the list of independent variables which can be continuous or binary.

The regression coefficients bᵢ can be exponentiated to give the change in odds for Y. Further, if it is determined that there are independent variables whose features are categorical, then tree ensembles for predicting the outcome may be used.

Returning to FIG. 4, the step 440 shows an example of the initial schedule being updated such that the order of the tests has been changed. For example, in the above example it may be considered that the patient now has a stress test that needs to be performed and the stress test either has a higher priority than the x-ray or the stress test has a shorter waiting time than the x-ray. Thus, the next event that is scheduled by the scheduling unit 101 is the stress test.

It should be understood that the step 440 may encompass multiple scenarios that are experienced by the patient. In the example provided above, the scenario is described that a new test (e.g., the stress test) is to be performed and this new test needs to be scheduled and that functionality is performed in step 440. However, another scenario may be that no new test is scheduled, but the next event that is scheduled based on the initial schedule has a waiting time that makes for an inefficient use of the patient's time. In such a situation, in step 440, the scheduling unit 101 may reschedule event(s) that were initially subsequent to the next event, but would now make the patient's visit more efficient by moving the event(s) ahead in the schedule. Thus, in this example, no new events have been added to the schedule, but the scheduling unit 101 makes a determination that the patient's schedule should be modified to produce a more efficient schedule.

In another example, it may not be the patient's schedule that is inefficient, but the use of the hospital resources that are being used inefficiently. For example, a particular department that the patient is scheduled to visit later in the day may have a light loading at the present time (e.g., due to a cancellation, etc.) and while it may still be efficient for the patient to go to the next scheduled event, the scheduling unit 101 may balance the load on the different hospital departments by rescheduling the next event for the patient to the more lightly loaded department. Thus, in this example, the rescheduling that occurs in step 440 is based on the use of the hospital resources. Another example of rescheduling based on hospital resources was described above, e.g., where the hospital had multiple labs/stations to perform the same functionality (e.g., x-rays, MRIs, etc.).

Examples of algorithms for scheduling and rescheduling have been provided above and these same algorithms may be used for any of these described examples. As also described above, the system 300 will have access to various information about the patient (e.g., events) and the hospital resources (e.g., current wait times, etc.) to perform this scheduling/rescheduling functionality.

Returning to FIG. 4, once the scheduling/rescheduling of step 440 is performed, the scheduling unit 101 provides the updated schedule to the navigation unit 102 to provide the updated schedule and navigation guidance to the patient. This is shown in step 450 where the navigation unit 102 as displayed by the user device 320 directs the patient to the stress test laboratory. The navigation step 450 is similar to the navigation step 420 described above, except that the destination is different.

In step 460 the stress test is performed on the patient. Similar to the step 430, the report generating unit 103 will record the conversations of the patient and doctor and perform the various functionalities described above. In step 470, the report generating unit 103 is shown as generating the report for this patient for the visit 400. Again, an example of generating a report was provided above with reference to step 430. In step 470 the same functionality may be performed to record the interaction between the patient and the caregiver and make recommendations for additional tests and/or follow-up events.

It should be noted that the visit 400 illustrated by FIG. 4 may not include all the events for the patient for this visit 400. For example, the patient may still need to have the x-ray and follow-up visit to the orthopedic doctor. Moreover, the addition of the stress test, may also trigger a new event such as a follow-up with a cardiologist. These additional events may be scheduled for the current visit 400, or depending on the level of severity and the hospital resources, the patient may be scheduled for a future visit.

It should be noted that FIGS. 5-7 call out various exemplary components for the scheduling unit 101, navigation unit 102 and report generating unit 103, respectively. However, these exemplary components are not required to be included within the respective units 101-103. That is, the units 101-103 may access the described functionality for the component by accessing a different device and/or system. To provide a specific example, the navigation unit 102 is described as including a map database 640. It is not required that the map database be a component of the navigation unit 102. Rather, the navigation unit 102 may simply access the map data that is stored on another system (e.g., one of the hospital data systems 330), as needed. In addition, the functionality described for any of the units 101-103 may be implemented as a functionality in another one of the units. That is, the delineation of the functionality of the units 101-103 is for the purpose of logically grouping the functionalities, but it is not necessary to implement the functionalities along these logical groupings.

FIG. 8 shows an exemplary user device 320 of the system 300 of FIG. 3. Specifically, the user device 320 is configured to execute a plurality of applications that perform some or all of the functionalities described above. As described above, the user device 320 is a portable device that may be provided to the patient and or a person accompanying the patient. The user device 320 may represent any electronic device that is configured to perform wireless functionalities. For example, the user device 320 may be a portable device such as a smartphone, a tablet, a phablet, a laptop, a wearable, etc. The user device 320 may be configured to perform cellular and/or WiFi functionalities. The user device 320 may include a processor 810, a memory arrangement 820, a display device 830, a speaker 840, a microphone 850, a transceiver arrangement 860 including a transmitter 865a and a receiver 865b, and other components 870. The other components 870 may include, for example, additional I/O components (a keyboard, a mouse, a keypad, a touchscreen), a battery that provides a limited power supply, a data acquisition device, ports to electrically connect the user device 320 to other electronic devices, etc. The functionality provided by some of these components (e.g., the microphone 850, the display device 830) has been described in detail above.

The processor 810 may be configured to execute a plurality of applications of the user device 320. For example, the applications may include the functionality associated with the navigation unit 102 as described above. It should be noted that the above applications being described as an application (e.g., a program) executed by the processor 810 is only exemplary. The functionality associated with the applications may also be represented as a separate incorporated component of the user device 320 or may be a modular component coupled to the user device 320, e.g., an integrated circuit with or without firmware. For example, the processor 810 may be a hardware component that comprises circuitry necessary to interpret and execute electrical signals fed into the system 300. Examples of processors include central processing units (CPUs), control units, microprocessors, etc. The circuitry may be implemented as an integrated circuit, an application specific integrated circuit (ASIC), etc. The exemplary embodiments may be implemented in any of these or other configurations of a user device 320.

The memory arrangement 820 may be a hardware component configured to store data related to operations performed by the UE 110. For example, the memory arrangement 820 may store data related to the functionality of the navigation unit 102 or the recording unit 710. The memory arrangement 820 may be any type of semiconductor memory, including volatile and non-volatile memory. Examples of non-volatile memory include flash memory, read only memory (ROM), programmable ROM (PROM), erasable PROM (EPROM) and electrically erasable PROM (EEPROM)se. Examples of volatile memory include dynamic random-access memory (DRAM), and fast CPU cache memory, which is typically static random-access memory (SRAM).

The display device 830 may be a hardware component configured to show data to a user. The display device 830 may be, for example, a liquid crystal display (LCD) device, a light emitting diode (LED) display, an organic LED (OLED) display, a plasma display panel (PDP), etc. Those skilled in the art will understand that the functionalities of the user interface and the display may be implemented in a single hardware component. For example, a touchscreen device may be used to implement both the display and the user interface.

The transceiver 860 may be a hardware component configured to wirelessly transmit data via the transmitter 865a and wirelessly receive data via the receiver 865b. The transceiver 860 may enable communication with the hospital network (e.g., WiFi network, WLAN, etc.) or with other electronic devices directly. The transceiver 325 may operate on a variety of different frequencies or channels (e.g., set of consecutive frequencies). Thus, an antenna (not shown) coupled with the transceiver 325 may enable the transceiver 325 to operate on these frequency bands.

FIG. 9 shows an exemplary server device 310 of the system 300 of FIG. 3. Specifically, the server device 310 is configured to execute a plurality of applications that perform some or all of the functionalities described above. As described above, the server device 310 is a backend processing device that communicates with the various hospital data systems 330 and the user device 310. The server device 310 may represent any electronic device that is configured to perform the above described functionalities. For example, the server device 310 may be a server computing device, a network arrangement device, a network host device, etc. The server device 310 may include a processor 910, a memory arrangement 920, a transceiver arrangement 960 including a transmitter 965a and a receiver 965b, and other components 970. Each of these components was described above for the user device 320 with reference to FIG. 8. The components of the server device 310 may be considered to be similar to the corresponding components of the user device 320.

FIG. 10 shows a further exemplary method 1000 for guiding a patient in a hospital according to various exemplary embodiments described herein. In step 1010, the patient arrives at the hospital and registers at hospital reception. As described above, based on information received from the patient such as an identification, the hospital staff or the system 300 may generate an initial list of events for the patient in step 1020.

In step 1030, the system 300, via the scheduling unit 101, will generate the initial schedule for the patient. This initial schedule will be provided to the navigation unit 102 that will navigate the patient to the next scheduled event in step 1040.

In step 1050, the user device 320, executing the report generating unit 103, will record the interaction between the patient and the caregiver. This recording of the interaction will facilitate the updating of the patient's EMR based on the interactions. This may also cause additional events to be added to the patient's list of events.

In step 1060, the system 300 will determine if any new events are to be added to the patient's list of events. If there are one or more events to be added, the method 1000 returns to step 1020 to add the new events to the list. The method 1000 may then continue back through the steps 1030-1050 where the new event(s) are scheduled and performed.

If in step 1060 there are no new events to be added to the list of events, the method 1000 continues to step 1070 to determine if the current schedule needs to be revised. As described above, there may be various reasons that may trigger a rescheduling even though no new events have been added (e.g., back-up at a next scheduled event, rescheduling to more effectively use patient's time, to balance load on hospital resources, etc.). If a rescheduling is required, the method 1000 proceeds back to step 1030 where the remaining events are rescheduled and the method 1000 continues back through the steps 1040-1060.

If in step 1070 no rescheduling is required, the method 1000 continues to step 1080 to determine if there are any events left to perform on the current schedule. If there are events that are left to be performed, the method 1000 continues back to step 1040 to navigate the patient to the next scheduled event and the method 1000 continues back through the steps 1050-1070. If there are no additional scheduled events, the patient's visit is complete and the method 1000 may end.

Those skilled in the art will understand that the above-described exemplary embodiments may be implanted in any number of manners, including, as a separate software module, as a combination of hardware and software, etc. Further, those skilled in the art will understand that the above-described exemplary embodiments may be used separately or in combination.

It is noted that the claims may include reference signs/numerals in accordance with PCT Rule 6.2(b). However, the present claims should not be considered to be limited to the exemplary embodiments corresponding to the reference signs/numerals.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

1. A method for guiding a patient in a hospital, comprising the steps of:
scheduling events for a patient based on a scheduling model and at least one scheduling parameter;
providing navigation information to the patient based on the scheduling of the events to enable performing the events; and
generating a report of the events.

2. The method as claimed in claim 1, wherein the events include one of a lab test or a consultation with physician.

3. The method as claimed in claim 2, wherein the events include one or more events of the same type or of different type or combination thereof.

4. The method as claimed in claim 1, wherein the events occur at one or more locations in a hospital.

5. The method as claimed in claim 1, wherein the at least one scheduling parameter includes one of a resource availability, a time for performing the event, a distance, a movement, a waiting time, a criticality of the event, a patient profile, clinical data, or a clinical condition.

6. The method as claimed in claim 1, wherein the scheduling model is a linear regression model.

7. The method as claimed in claim 1, wherein the navigation information includes one of a location of facilities, a location of a doctor's station, a navigation path between facilities, a time to navigate, a resource availability and a time of availability, a sequence of events, or a sequence of navigation.

8. The method as claimed in claim 1, wherein the generating the report includes one of recording an interaction between the patient and the resource.

9. The method as claimed in claim 1, wherein the generating the report includes providing a recommendation on events to the patient.

10. The method as claimed in claim 9, wherein providing the recommendation includes one of altering the events or altering the schedule of events.

11. The method as claimed in claim 9, wherein the generating the report includes providing the recommendation based on one of a patient profile, a clinical condition of the patient, another patient profile or another clinical condition related to the patient profile and clinical condition.

12. A system for guiding a patient in a hospital, comprising:
a scheduling unit configured to schedule events for a patient based on a scheduling model and at least one scheduling parameter;
a navigation unit configured to provide navigation information to the patient based on the scheduling of the events to enable performing the events; and
a report generating unit configured to generate a report of the events.

13. The system as claimed in claim 12, wherein the scheduling unit, the navigating unit and the report generating unit are implemented via a processing unit.

14. The system as claimed in claim 12, wherein the scheduling model is a linear regression model.

15. The system as claimed in claim 12, wherein the navigation unit has one of audio or video capability and a location identifier that includes one of a Global Positioning System (GPS) or WiFi location detection.

16. A system, comprising:
a server device configured to extract information from one of a patient record and a hospital resource to generate a list of events for a patient, the server device further configured to generate a schedule for the patient based on the list of events; and
a user device configured to receive the schedule from the server device and navigate the patient to a first event on the schedule.

17. The system of claim 16, wherein the user device is further configured to record an interaction between the patient and a caregiver at the first event.

18. The system of claim 17, wherein the server device is configured to receive the recorded interaction and generate a report for the first event.

19. The system of claim 18, wherein the server device is further configured to one of recommend a new event or remove one of the events based on the recorded interaction.

20. The system of claim 19, wherein the server device is further configured to modify the schedule to account for the one of the new event or the removed one of the events.
